(19) United States
(12) Patent Application Publication
(10) Pub. No.: US 2010/0034050 A1
(43) Pub. Date: Feb. 11, 2010

(54) Apparatus and Method for Cultivating Algae

(76) Inventors: Gary Erb, Crystal Lake, IL (US); David Ross Peterson, Dixon, IL (US)

Correspondence Address: MILLER, MATTHIAS & HULL, ONE NORTH FRANKLIN STREET, SUITE 2350, CHICAGO, IL, 60606, US

(21) Appl. No.: 12/189,468

(22) Filed: Aug. 11, 2008

(51) Int. Cl. B01F 3/00; C12M 1/06

(52) U.S. Cl. 366/342; 435/292.1

(57) ABSTRACT

Apparatus and methods for cultivating photosynthetic organisms, such as algae, in a bioreactor may include a bioreactor having a primary tank. Light and carbon dioxide are provided in the tank sufficient to promote algae growth. The algae is agitated inside the tank to increase the amount of algae receiving sufficient light exposure inside the tank. Agitation may be provided by a closed loop circulation system or a mixer having a plurality of rotating blades. The gas source may be positioned and oriented with respect to the light source to keep the light source free of adhered material.

### FIELD OF THE DISCLOSURE

This disclosure generally relates to an apparatus and method for growing photosynthetic microorganisms, and more particularly for growing algae. Certain embodiments also relate to a system for producing useful products from algae, such as biofuels and protein.

### BACKGROUND OF THE DISCLOSURE

A variety of methods and technologies exist for cultivating and harvesting biomass such as, for example, mammalian, animal, plant, and insect cells, as well as various species of bacteria, algae, plankton, and protozoa. These methods and technologies may include open-air systems and closed systems. Algal biomasses, for example, are often cultured in open-air systems (e.g. ponds, lakes, raceway ponds, and the like) that are subject to contamination. These open-air systems are further limited by an inability to substantially control the various process parameters (e.g., temperature, incident light intensity, flow, pressure, nutrients, and the like) involved in cultivating algae.

Alternatively, algae may be cultivated in closed systems called bioreactors. Closed systems allow for better control of the process parameters but are typically more costly to set up and operate. In addition, conventional closed systems are limited in their ability to provide sufficient light to sustain dense populations of photosynthetic organisms cultivated within.

Biomasses have many beneficial and commercial uses including, for example, as pollution control agents, fertilizers, food supplements, cosmetic additives, pigment additives, and energy sources just to name a few. For example, algal biomasses are used in wastewater treatment facilities to capture fertilizers. Algal biomasses are also used to make biofuels.

Bioreactors used for growing photosynthetic organisms typically employ a constant intensity light source. A key factor for cultivating biomasses such as algae in bioreactors is provided in controlling the light necessary for the photosynthetic process. If the light intensity is too high or the exposure time to long, growth of the algae is inhibited. Moreover, as the density of the algae cells in the bioreactors increases, algae cells closer to the light source limit the ability of those algae cells that are further away from absorbing light. This factor has limited the size of conventional, closed bioreactors.

Commercial acceptance of bioreactors is dependent on a variety of factors such as cost to manufacture, cost to operate, reliability, durability, and scalability. Commercial acceptance of bioreactors is also dependent on their ability to increase biomass production, while decreasing biomass production costs. Accordingly, it may be desirable to provide a bioreactor capable of operating at a commercial scale.

### SUMMARY OF THE DISCLOSURE

A bioreactor for cultivating photosynthetic organisms includes a primary tank having a sidewall oriented along a longitudinal axis, a mixer disposed inside the tank, a light source disposed inside the tank, and a sparger disposed inside the tank and adapted to fluidly communicate with a source of carbon dioxide.

According to additional aspects, a bioreactor is provided for cultivating photosynthetic organisms disposed in a fluid. The bioreactor includes a primary tank having a sidewall oriented along a longitudinal axis and defining an inlet end and an outlet end. An inlet pipe is coupled to the primary tank inlet end and an inlet valve is disposed in the inlet pipe and movable between open and closed positions. An outlet pipe is coupled to the primary tank outlet end and an outlet valve is disposed in the outlet pipe and movable between open and closed positions. A recirculation pipe has a first end coupled to the primary tank inlet end and a second end coupled to the primary tank outlet end, and a recirculation pump is disposed in the recirculation pipe. A light source is disposed in at least one of the primary tank and recirculation pipe, and a gas source disposed inside the tank.

According to further aspects, a method for agitating photosynthetic organisms in a fluid disposed within a bioreactor includes providing a primary tank including a sidewall oriented along a longitudinal axis, a mixer disposed inside the tank, a light source inside the tank, and a gas source inside the tank. The method includes operating the mixer to create a complex fluid flow pattern inside the primary tank, in which the complex fluid flow pattern includes at least a first and second fluid path sections, wherein the first fluid path section flows substantially in a first direction along the longitudinal axis and the second fluid path section flows substantially in a second, opposite direction along the longitudinal axis.

### BRIEF DESCRIPTION OF THE DRAWINGS

For a more complete understanding of the disclosed methods and apparatus, reference should be made to the embodiments illustrated in greater detail on the accompanying drawings, wherein;

FIG. 1 is a schematic illustration of a bioreactor according to the present disclosure having a horizontally aligned primary tank;

FIG. 2 is a schematic illustration of an alternative embodiment of a bioreactor having a vertically oriented primary tank;

FIG. 3 is a schematic illustration of an algae cultivation and reproduction system including the bioreactor of FIG. 2;

FIGS. 4A and 4B are schematic plan views illustrating alternative impeller in the bioreactor of FIG. 2;

FIG. 5 is an enlarged plan view of a portion of an impeller usable in the bioreactor of FIG. 2;

FIGS. 6A and 6B are side elevation views, in cross-section, of the impeller of FIG. 5 taken along lines 6a-6a and 6b-6b respectively;

FIGS. 7A and 7B are schematic perspective views of alternative turbine blades usable in the bioreactor of FIG. 2;

FIGS. 8A and 8B are schematic perspective views of a further impeller embodiment having primary and outer sections that are adjustable with respect to each other;

FIGS. 9A and 9B are perspective schematic illustrations of a yet another impeller embodiment having inner, primary, and outer sections that are adjustable with respect to one another;

FIGS. 10A-10D are schematic illustrations showing possible fluid flow paths inside the primary tank;

FIGS. 11A and 11B are schematic illustrations showing fluid-filled tubing surrounding a horizontally orientated primary tank, respectively;

FIG. 12A is a schematic side elevation view, in cross-section, of a bioreactor employing a set of rotating blades and a set of non-rotating blades;

FIG. 12B is a schematic plan view of the bioreactor of FIG. 12A;

FIG. 13 is an enlarged schematic perspective view of a single rotating blade and a single non-rotating blade from FIG. 12;

FIG. 14 is an enlarged schematic perspective view of an alternative embodiment of rotating and non-rotating blades;

FIG. 15 is an enlarged schematic perspective view of an alternative embodiment of rotating and non-rotating blades;

FIG. 16 is an enlarged schematic perspective view of an alternative embodiment of rotating and non-rotating blades; and

FIG. 17 is a schematic perspective view of a bioreactor employing curved light panels.

It should be understood that the drawings are not necessarily to scale and the disclosed embodiments are sometimes illustrated diagrammatically in partial views. In certain instances, details which are not necessary for an understanding of the disclosed methods and apparatus, or which render other details difficult to perceive, may have been omitted. It should be understood, of course, that this disclosure is not limited to the particular embodiments illustrated herein.

### DETAILED DESCRIPTION OF THE DISCLOSURE

A method and apparatus for cultivating algae is disclosed that uses waste and products from other systems as inputs. The algae produced by the method and apparatus contain polysaccharide, proteins, and lipids, which may be further processed into biodiesel, glycerin and mono sugars (which may be further fermented into ethanol and other alcohol products). The exemplary embodiments employ a closed bioreactor system to produce the algae. The system generally includes a primary tank for receiving a slurry of water and algae. The tank further includes a light source, means for mixing the fluid, a carbon dioxide, and a heat source. The light, fluid mixing, carbon dioxide, are controlled to produce an environment inside the tank that is conducive to producing and cultivating algae. The bioreactor increases algae yield by using a plurality of lights and/or agitating the fluid inside the tank.

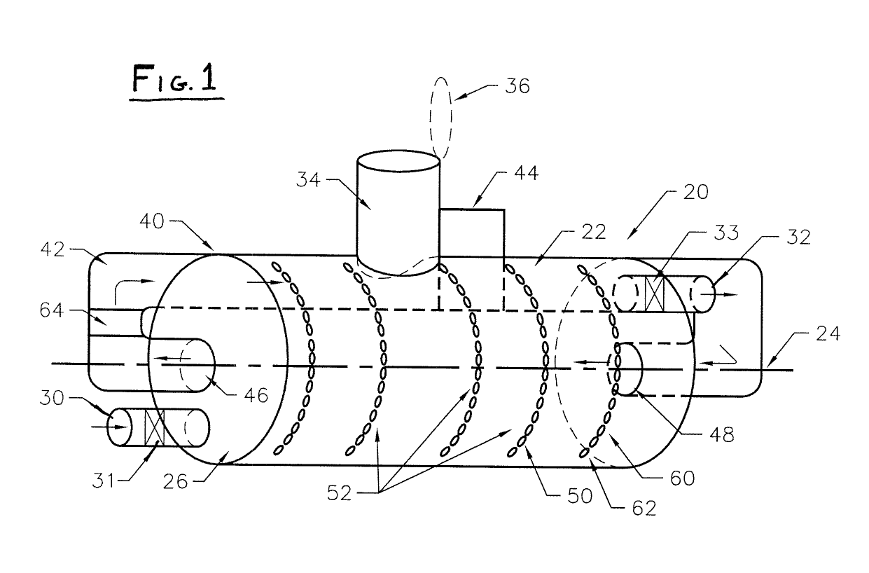

A first embodiment of a bioreactor is illustrated in FIG. 1. The bioreactor 20 generally includes a primary tank 22 disposed along a horizontal axis 24. The tank 22 may have any commercially feasible size. For example, when the tank 22 is cylindrical, it may have a diameter greater than one foot, and preferably five to eight feet or more, if sufficient space and soil depth are available. The length of the tank is also largely dependent on the available space and volume of algae cultivation desired. For example, the tank 22 may be twelve to twenty-two feet long or more. Similarly, the volume of the tank may be selected according to desired production rate and available space, such as approximately 1,500-5,000 gallons.

The primary tank 22 is preferably insulated, such as by surrounding the tank 22 with temperature controlled, fluid-filled tubing 23 as best shown in FIG. 11B. Additionally or alternatively, the primary tank 22 may be located partially or fully underground to provide natural insulation.

The primary tank 22 defines a first or inlet end 26 or a second or outlet end 28. An inlet pipe 30 is coupled to the first end 26 while an outlet pipe 32 is coupled to the outlet end 28. Inlet and outlet valves, 31, 33, are disposed in the inlet and outlet pipes 30, 32, respectively, to control fluid flow into and out of the primary tank 22. The primary tank 22 may further include an access hatch 34 and associated lid 36. The axis hatch 34 is sized to permit axis to the interior of the primary tank 22 for inspection and maintenance purposes. The lid 36 preferably forms an airtight seal when closed to help maintain the desired process parameters inside the tank 22 and to prevent gas from escaping from the tank.

The exemplary bioreactor 20 further includes a circulation system 40 that mixes and agitates the fluid inside the primary tank 22. In the illustrated embodiment, the circulation system 40 includes a circulation pipe 42 having a circulation pump 44 disposed therein. The circulation pipe 42 may have a first end 46 fluidly communicating with the inlet end of the primary tank 22 and a second end 48 fluidly communicating with the outlet end of the tank 22. The pump 44 may be orientated so that fluid flows through the circulation pipe from the first end 46 to the second end 48. It will be appreciated that this fluid flow agitates and mixes the fluid inside the tank thereby to expose different portions of the fluid through the interior tank surface.

The bioreactor 20 further includes a sparger 50 for introducing carbon dioxide into the primary tank 22. In the illustrated embodiment, a plurality of nozzles 52 are formed around a periphery of the tank 22 and are orientated to discharge into the tank interior. The nozzles 52 fluidly communicate with a carbon dioxide source (not shown). In operation, the nozzles 52 inject carbon dioxide gas into the interior, which is subsequently consumed during the photosynthesis process. The sparger 50 may also assist with agitating the algae slurry.

Carbon dioxide may be introduced into the primary tank 22 via other alternatives, should bubble lysis of the algae cells become a problem. Bubble lysis is the lysis of algae cells as bubbles of carbon dioxide burst. While a certain level of bubble lysis is inevitable inside the primary tank 22, a significant level of bubble lysis is detrimental to algae cultivation inside the tank 22. Should bubble lysis become an issue, the carbon dioxide may be injected into the algae slurry prior to introduction into the primary tank 22, thereby mitigating the amount of bubble lysis inside the tank.

The bioreactor 20 may also include a light apparatus 60 for providing light inside the primary tank 22. In the illustrated embodiment, the light apparatus 60 includes a plurality of individual lights 62 positioned along a periphery of a light interior. Alternatively, the lights 62 may be suspended at various positions inside the tank 22. Each light 62 may comprise an artificial light source such as an LED, or a natural light source such as a network of fiber optic wave guides coupled to a solar collector. The lights 62 are spaced throughout the tank primary tank 22 to increase the volume inside the tank that receives sufficient light to promote algae growth. The light apparatus 60 may additionally or alternatively include a light 64 positioned in the circulation pipe 42 which creates a light zone in the pipe 42 through which the algae slurry passes as it flows through the pipe 42.

In operation, the inlet valve 31 is opened to permit algae feed stock to be loaded into the primary tank 22 through the tank inlet pipe 30. The inlet and outlet valves 31, 33 are then closed to retain the algae feed stock in the primary tank 22. The light apparatus 60 and sparger 50 are operated to provide the desired amount of light and carbon dioxide in the tank 22 to create an environment suitable for growing and cultivating a particular type of algae. The algae feed stock is then agitated to increase the amount of algae receiving sufficient light from the light apparatus 60. Agitation is accomplished primary by operating the circulation system 40 and by the carbon dioxide bubbling through the liquid. Agitation displaces the slurry so that different portions of the algae are positioned adjacent the light apparatus 60, thereby improving algae cultivation and minimizing or eliminating putrefication of the algae. Additionally, the larger volume of fluid that can be processed in the tank acts as a buffer to maintain a more constant pH level.

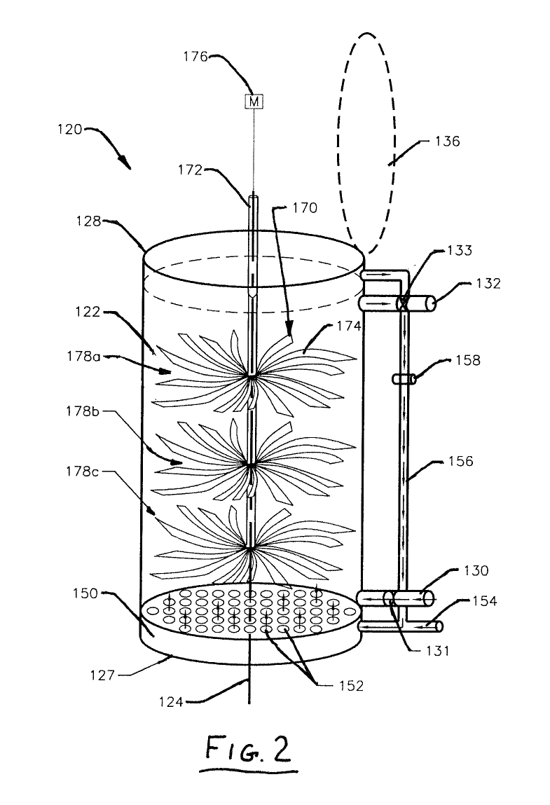

An alternative embodiment of a bioreactor 120 is illustrated in FIG. 2. The bioreactor 120 includes a primary tank 122 oriented along a vertical axis 124. The vertical primary tank 122 may have any commercially feasible size. For example, the tank 122 may have a diameter of approximate twelve to twenty feet, and a height of twenty to thirty feet, creating a corresponding volume of approximately 17,000 to 70,000 gallons. The above dimensions and volumes are merely exemplary, as the tank size may be scaled to the desired algae output rate and/or available space. The primary tank 122 includes a first or bottom end 127 and a second or top end 128. An inlet pipe 130 fluidly communicates with the interior of the tank 122 near the end bottom 126, while an outlet pipe 132 fluidly communicates with the interior of the tank 122 near the top end 128. Inlet and outlet valves 131, 133 are disposed in the inlet and outlet pipes 130 and 132 respectively. A lid 136 is removably coupled to the top end 128 of the primary tank 122 and is configured to form an airtight seal when attached to the tank 122. The primary tank 122 may be insulated, such as by fluid-filled tubing 123 as illustrated in FIG. 11B.

The exemplary bioreactor 120 includes a gas delivery system for introducing carbon dioxide into the primary tank 122. In the illustrated embodiment, a sparger 150 is disposed near the bottom end 126 of the primary tank 122 and includes a plurality of nozzles 152 for introducing carbon dioxide into the tank. The sparger 150 fluidly communicates with a gas inlet pipe 154 that is connected to a source of carbon dioxide (not shown). A gas recirculating pipe 156 has an inlet in fluid communication with the top end 128 of the primary tank 122 and an outlet in fluid communication with gas inlet pipe 154. A pump 158 is disposed in the gas recirculating pipe 156 to pull gas from the tank top end 128 and push gas to through the sparger 150.

The bioreactor 120 further includes a mixer 170 for agitating the algae feed stock inside the primary tank 122. As illustrated in FIG. 2, the mixer 170 includes a rotatable shaft 172 disposed inside the primary tank 122. A plurality of turbine blades 174 is coupled to the shaft 172. The shaft 172 is operatively coupled to a motor 176 which rotates the shaft 172 and attached blade 174. In operation of the blades 174 displaces the algae slurry inside the primary tank 122 so that the slurry is circulated throughout the tank. The turbine blades 174 may include groups of blades positioned at spaced elevations along the shaft 172 to form discrete sets of turbine blades 178a, 178b, 178c. The shaft 172 may further comprise segments that are supported for rotation in different directions, thereby to provide counter-rotating turbine blades. Other alternative blade configurations and styles are disposed below that are suitable for use in the bioreactor 120.

The turbine blades 174 may be configured to maximize fluid circulation inside the primary tank 122. As shown in FIG. 4a, turbine blades 174a comprise radial blades that are substantially linear. Each blade 174a is sized to extend from the shaft 172 to a point near the interior surface of the primary tank 122. As shown in FIG. 4b, the turbine blades 174b have a curved configuration.

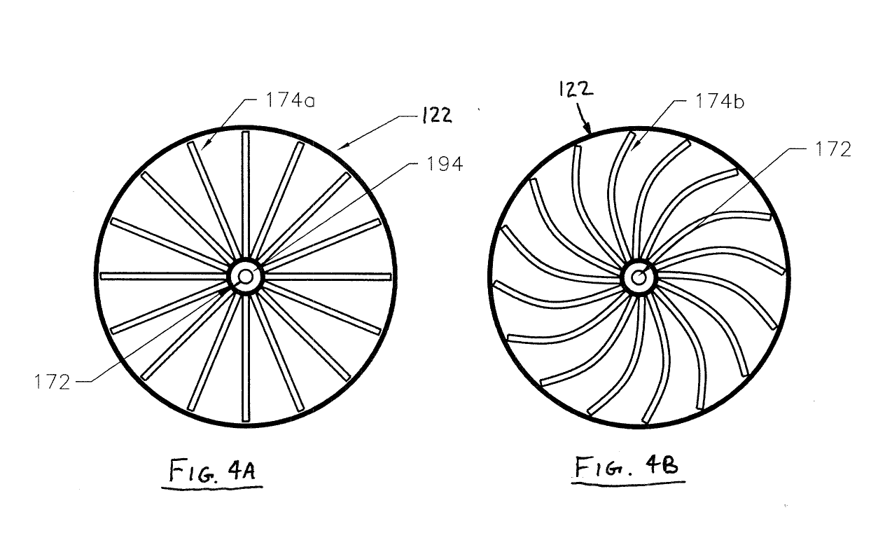

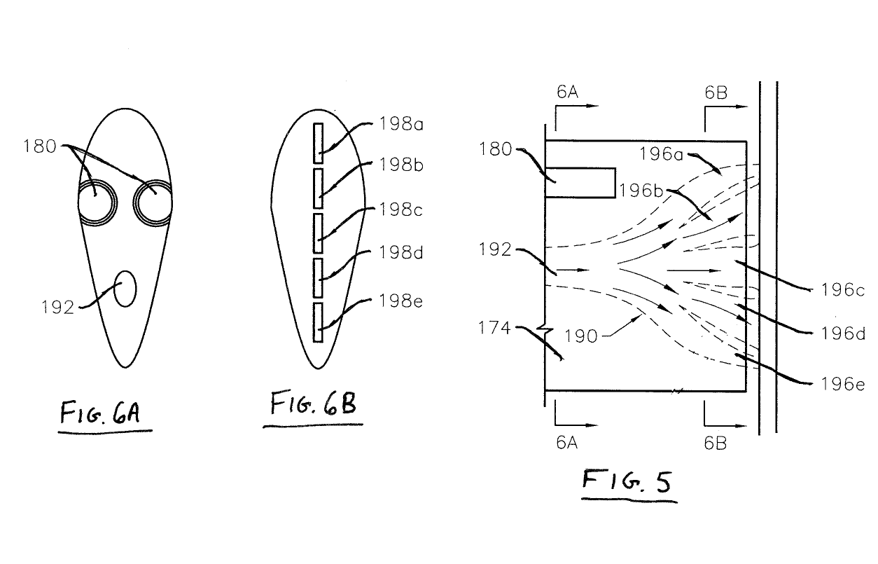

The turbine blades 174 may further includes means for illuminating the algae slurry as well as for dispensing carbon dioxide into the primary tank 122. An outer segment of turbine blade 174 is schematically illustrated at FIG. 5. A light source, such as an elongated light tube 180 is coupled to the turbine blade 174 and positioned near an exterior surface thereof. The turbine blade 174 may include two or more light tubes 180 disposed on opposed surfaces of the blade as shown in FIGS. 6a. Additionally, a gas conduit 190 may extend longitudinally through the turbine blade 174. The gas conduit may include a primary conduit section 192 having an inlet in fluid communication with the central gas conduit 194 formed through the shaft 172. The primary segment 192 fluidly communicates with a plurality of branches 196a-e which form outlets 198a-e (FIG. 6b) for discharging carbon dioxide into the primary tank 122. Each turbine blade 174, or a sufficient number thereof, used in the primary tank 122 may include a light tube 180 so that a plurality of lights are disposed throughout the tank. Additionally, each turbine blade 174 may include a gas conduit 190 to introduce carbon dioxide simultaneously throughout the tank.

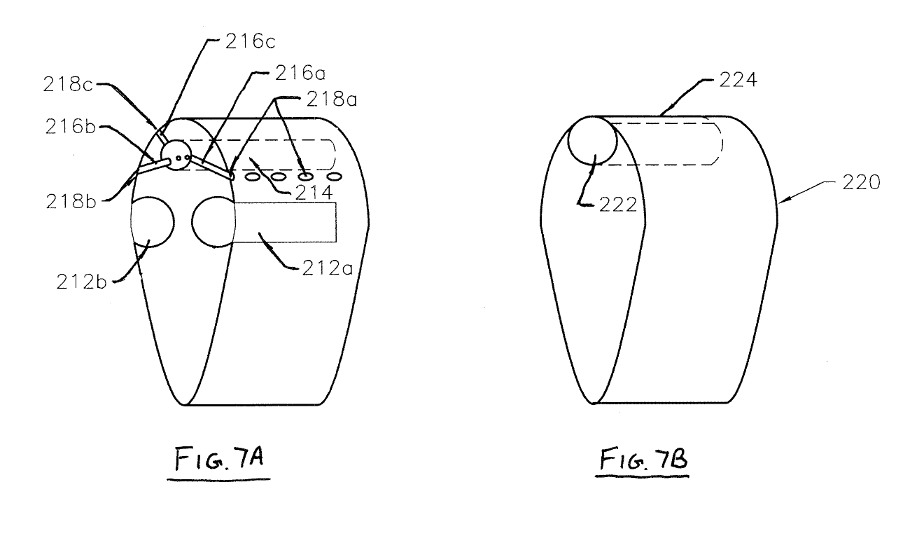

FIGS. 7A and 7B illustrate alternative turbine blade arrangements. In FIG. 7A, a turbine blade 210 includes two light tubes 212a, 212b extending along opposite sides of an exterior surface thereof. A gas conduit 214 extends through the turbine blade 210 and fluidly communicates with a source of carbon dioxide (not shown). Branches 216a-c extend outwardly from the gas conduit 214 to form gas outlets 218a-c. At least some of the outlets, such as outlets 218a and 218b, are positioned and oriented to discharge carbon dioxide toward the light tubes 212a, 212b. For example, outlet 218a is located near the light tube 212a. The branch 216a leading to outlet 218a is angled toward the light tube 212a so that gas exiting the outlet 218a initially flows toward the light tube 212a. The gas flow helps clear algae material from the turbine blade 210 in the vicinity of the light tube 212a, thereby increasing the effective area it illuminates inside the primary tank.

FIG. 7B illustrates a simplified turbine blade 220 that omits the carbon dioxide gas conduit. Instead, the turbine blade 220 includes an elongate light source 222 positioned at a leading edge 224 of the blade 220.

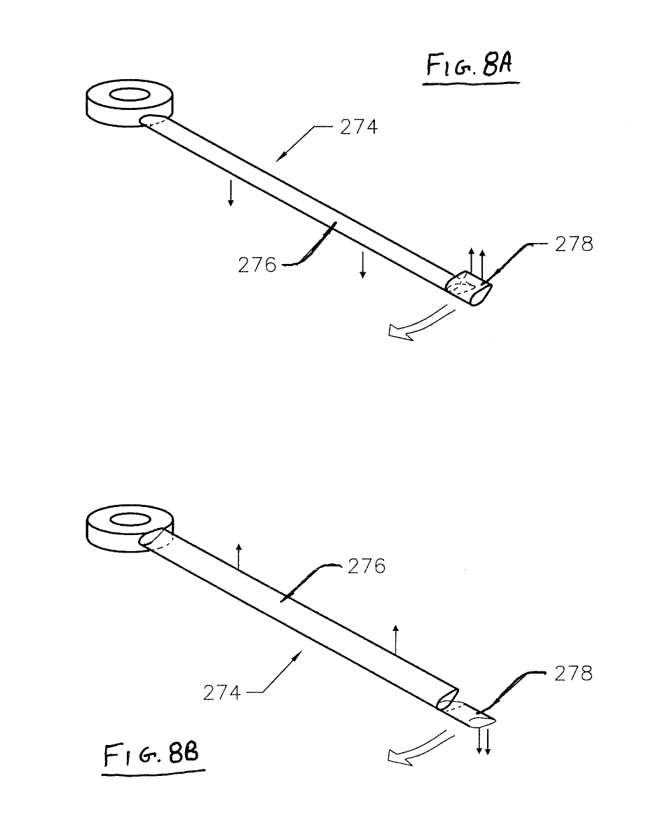

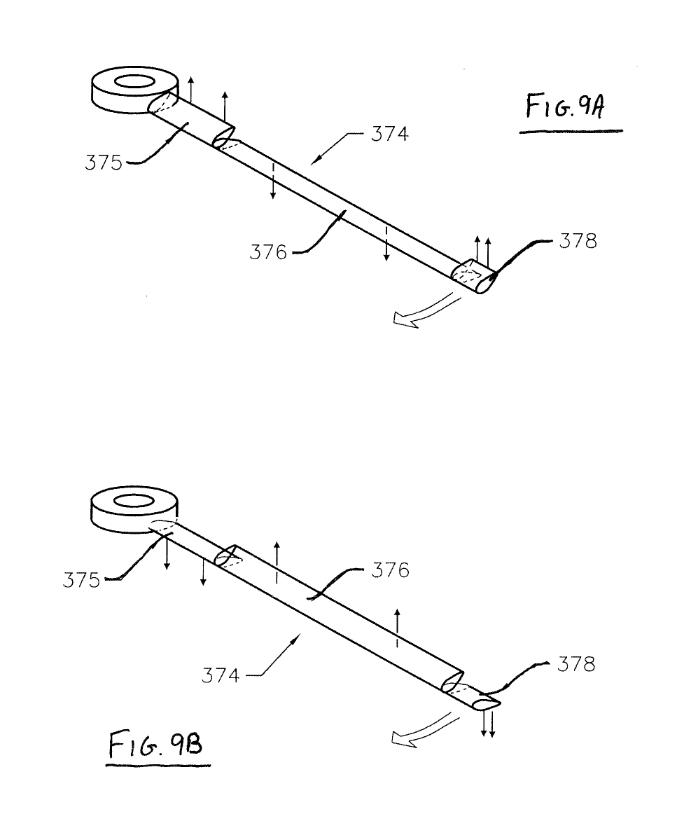

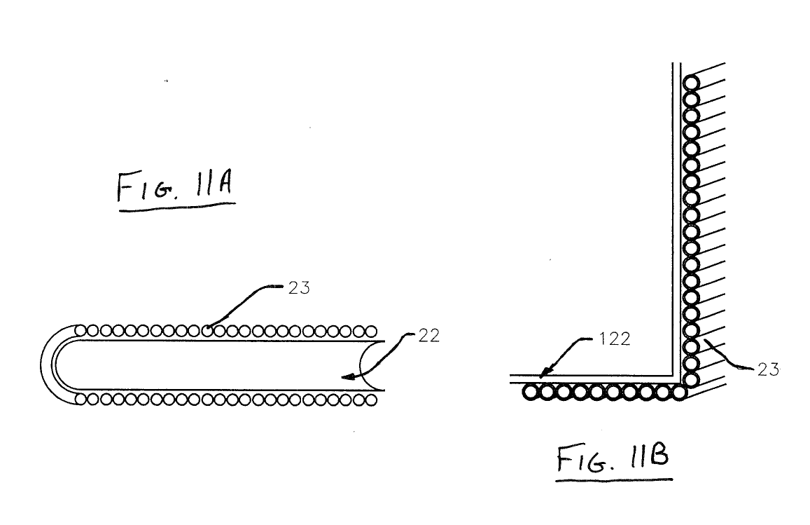

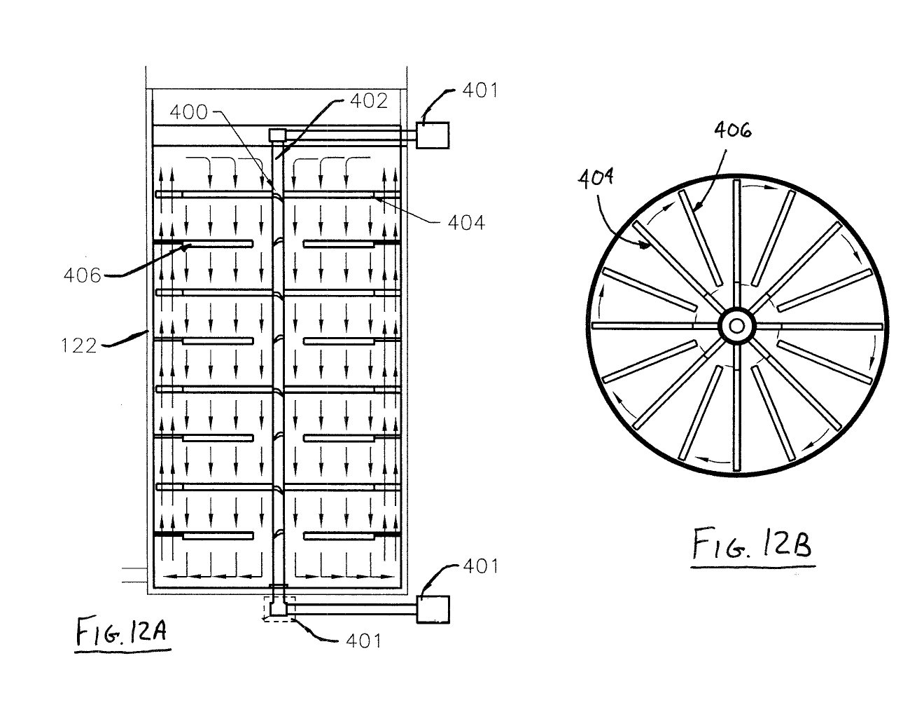

The turbine blades may include separate sections which permit complex fluid flow patterns to be formed inside the primary tank 122. In the embodiment illustrated in FIGS. 8A and 8B, a turbine blade 274 includes a primary section 276 positioned near a shaft 272 and an outer section 278. At least one of the primary and outer sections is oriented differently with respect to a remainder of the blade 274 to create different fluid flow velocities and/or directions. As shown in FIG. 8A, the outer section 278 is rotated upward with respect to the primary section 276 to generate upward fluid flow near the surface of the primary tank 122. A schematic of this complex fluid flow path is provided at FIG. 10A, which shows an inner fluid path section 280a flowing downward and an outer fluid path section 282b flowing upward. The downward flow direction of the larger, inner fluid path section 280a is counter to the flow of carbon dioxide from the sparger located at a bottom of the primary tank 122, thereby increasing the degree to which the algae slurry is agitated. Conversely, FIG. 8B shows the primary section 276 rotated upwardly with respect to the outer section 278 to create resulting upward and downward fluid flows respectively. A schematic of this fluid flow path is provided at FIG. 10B, which shows an inner fluid path section 280b flowing upward and an outer fluid path section 282b flowing downward.

The velocities of each fluid path section may be altered by the relative sizes of the primary and outer sections of the rotating blades. In the embodiments illustrated in FIGS. 8A and 8B, the blade primary section is significantly larger than the blade outer section. As a result, the outer fluid path section has a relatively higher velocity than the inner fluid path section, which may advantageously remove adhered algae matter along the inner surface of the primary tank 122 as well as any light sources located thereon.

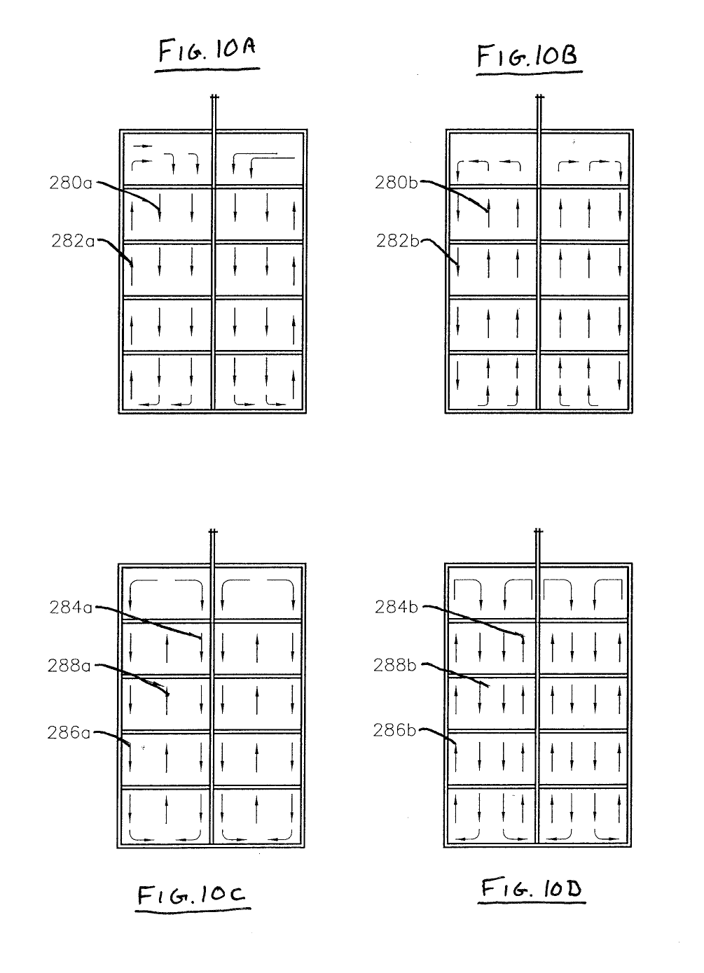

FIGS. 9A and 9B illustrate turbine blades 374 having three different sections. As shown in FIG. 9A, the turbine blade 374 includes an inner section 375, a primary section 376 and an outer section 378. As shown in FIG. 9A, the inner and outer sections 375, 378 are rotated upwardly with respect to the primary section 376 which will result in upward flows near the center and interior surface of the primary tank 122, while the primary section 376 will generate a downward fluid flow. A schematic of this fluid flow path is provided at FIG. 10C, which shows inner and outer fluid path sections 284a, 286a flowing downward and an intermediate fluid path section 288a flowing upward. Conversely, as shown in FIG. 9B, the primary section 376 has been rotated upwardly with respect to the inner and outer sections 375, 378 to generate the opposite fluid flow directions. A schematic of the resulting fluid flow path is provided at FIG. 10D, which shows inner and outer fluid path sections 284b, 286b flowing upward and an intermediate fluid path section 288b flowing downward.

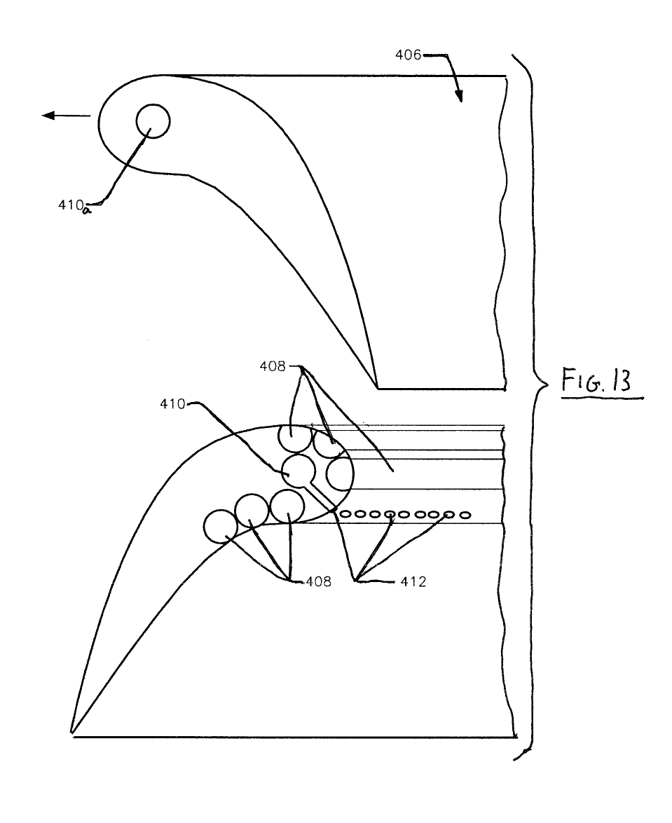

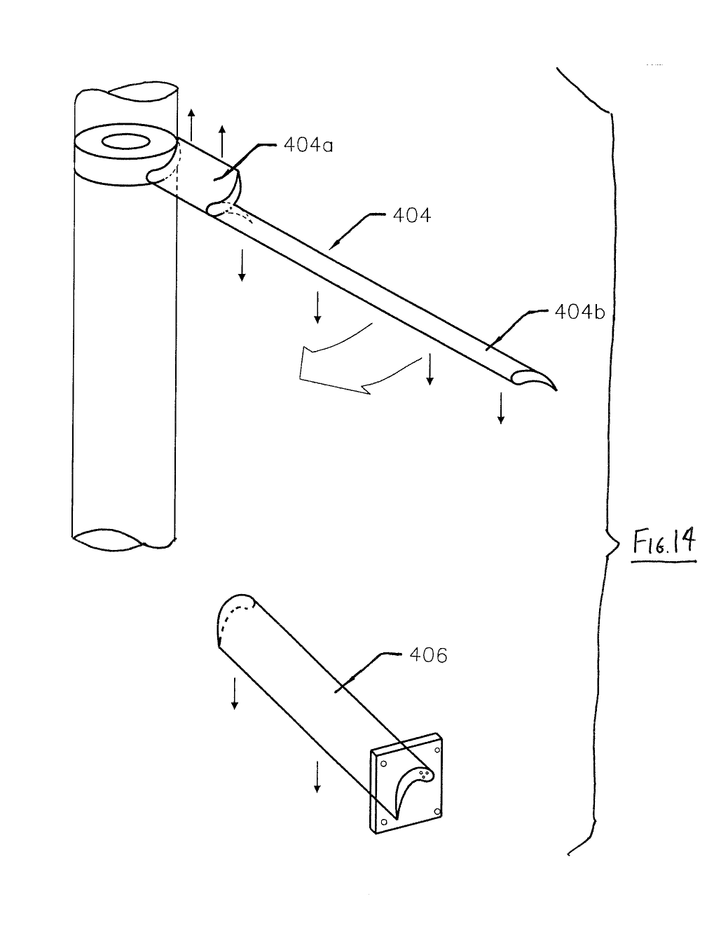

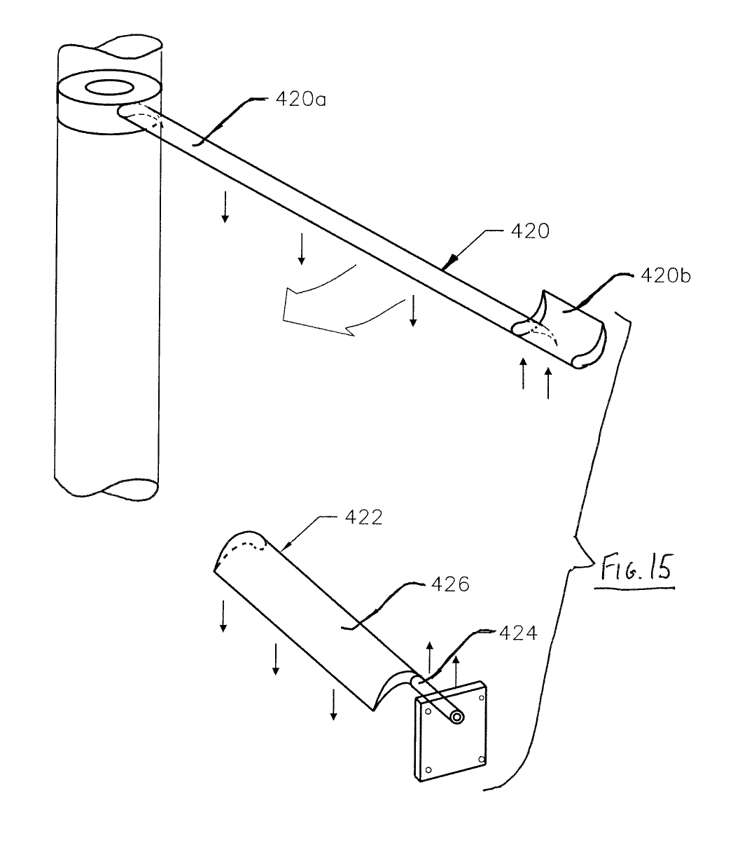

An alternative embodiment having both rotating and non-rotating blades is illustrated in FIGS. 12A, 12B, and 13. As best shown in FIG. 12A, the primary tank 122 includes a mixer 400 that includes a shaft 402 coupled to a motor 401. FIG. 12A illustrates three alternative positions for the motor: (1) position A near a top of and radially offset from the shaft 402; (2) position B near a bottom of and radially offset from the shaft 402; and (3) position C near a bottom of and axially aligned with the shaft 402. The shaft 402 is further coupled to a plurality of rotating blades 404. Additionally, non-rotating blades 406 extend inwardly from the interior surface of the tank 122. The rotating blades 404 define a rotating blade path that at least partially overlaps the non-rotating blades 406 in an axial direction. Stated another way, the rotating and non-rotating blades 404, 406 may be axially aligned so that an axial fluid flow stream may encounter both the rotating and non-rotating blades 404, 406 during operation. Additionally, the non-rotating blades 406 may be configured so that they permit removal of the rotating blades 404 from the primary tank 122, as shown in FIG. 12B, thereby facilitating cleaning, repair, or replacement of the rotating blades.

Both the rotating and non-rotating blades 404, 406 may be configured to induce a desired fluid flow pattern inside the tank 122. As shown in FIG. 13, both sets of blades 404, 406 have an air foil configuration. Each non-rotating blades 406 may incorporate one or more light sources 408, as well as a carbon dioxide conduit 410, thereby simplifying these systems by eliminating the need to route them through a rotating shaft. The carbon dioxide conduit 410 fluidly communicates with outlet orifices 412 positioned near the light sources 408, so that gas flow exiting the outlet orifices 412 will clear algae material from the surfaces of the light sources 408. Additionally or alternatively, each rotating blades 404 may include a carbon dioxide conduit 410a.

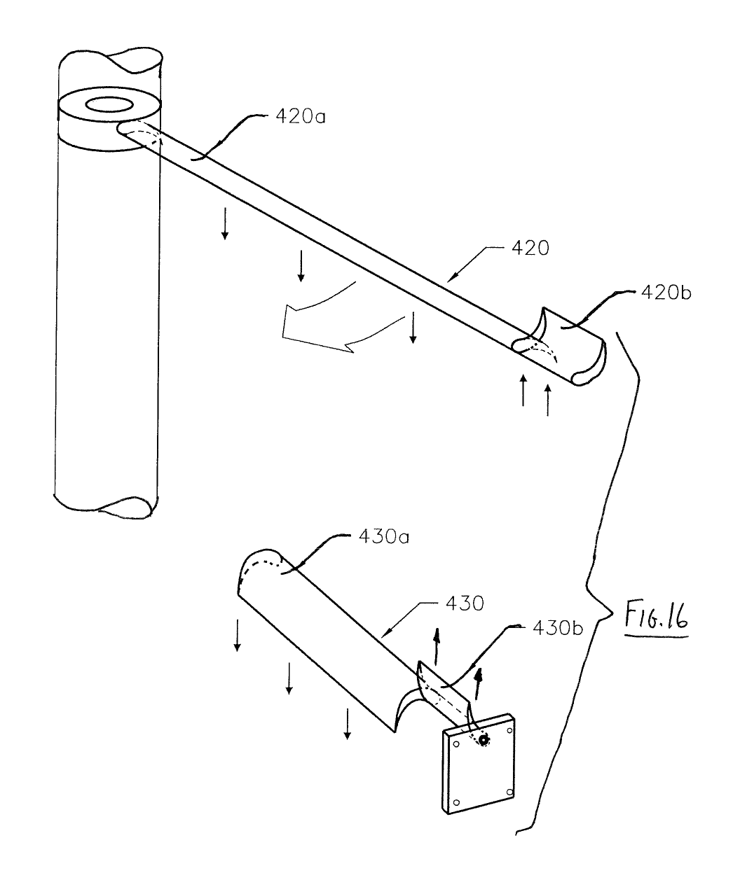

The rotating blades 404 may have inner and outer sections 404a, 404b to direct fluid flow in opposite directions as the blade 404 rotates. As shown in FIG. 14, the inner section 404a is configured to generate an upward fluid flow adjacent the shaft 402, while the outer section 404b is configured to generate a downward fluid flow. The non-rotating blades 406 may also be configured as shown to generate the downward fluid flow.

FIG. 15 shows an alternative embodiment similar to that shown in FIG. 14. In FIG. 15, a rotating blade 420 has an inner section 420a configured to create a downward fluid flow and an outer section 420b configured to create an upward fluid flow. A fixed blade 422 includes an outer post section 424 that is axially aligned with the rotating blade outer section 420b and has a profile that creates little resistance to the upward fluid flow generated by the rotating blade outer section 420b. The fixed blade 422 also has an inner section 426 that is axially aligned with the rotating blade inner section 420a and is configured to assist with generating a downward fluid flow in the corresponding portion of the tank.

FIG. 16 is a further alternative embodiment similar to that of FIG. 15. The embodiment of FIG. 16 uses the same rotating blade 420 as in FIG. 15. A fixed blade 430, however, includes an outer section 430b that is axially aligned with the rotating blade outer section 420b and is configured to assist with generating an upward fluid flow. The profile of the non-rotating blade outer section 430b also reduces the whirlpool effect generated by the rotating blades 420. An inner section 430a of the fixed blade is axially aligned with the rotating blade inner section 420a and is configured to assist with generating a downward fluid flow in a corresponding portion of the tank.

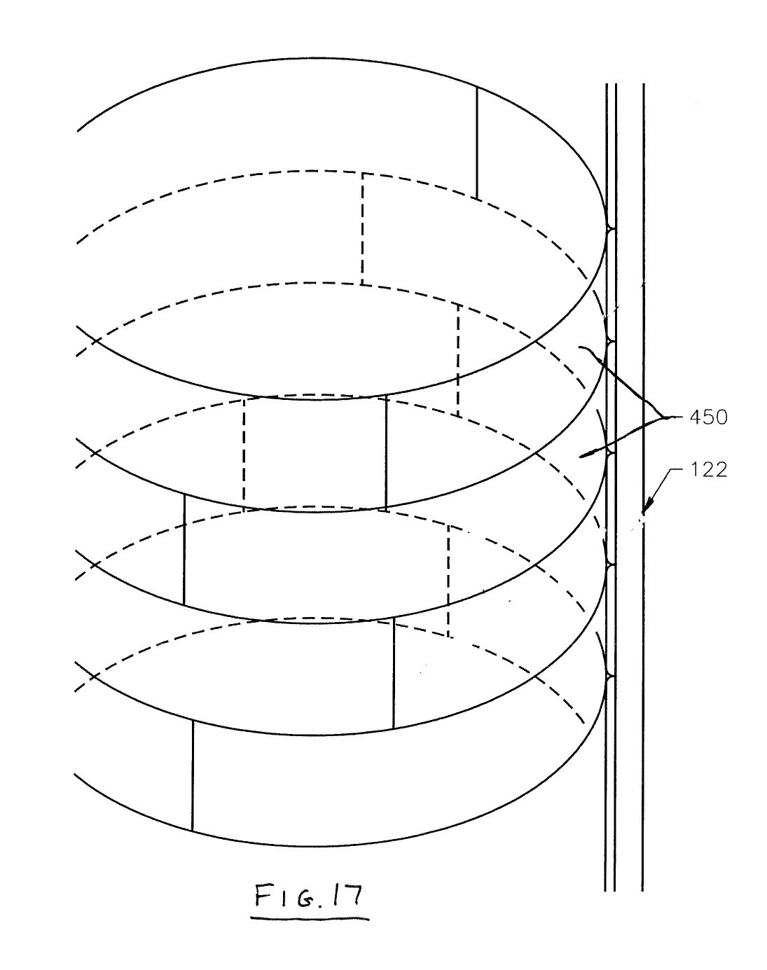

An alternative lighting source is illustrated in FIG. 17, in which curved lighting panels 450 line an interior surface of the primary tank 122. The curved lighting panels 450 may be formed using flexible light panels that substantially conform to the shape of the tank as they are applied to the tank interior surface, or may be rigidly formed with substantially the same radius as the tank and then attached to the tank interior surface. The lighting panels 450 allow the entire interior surface of the tank side wall to be used as a light source, thereby increasing the amount of light generated inside the tank 122.

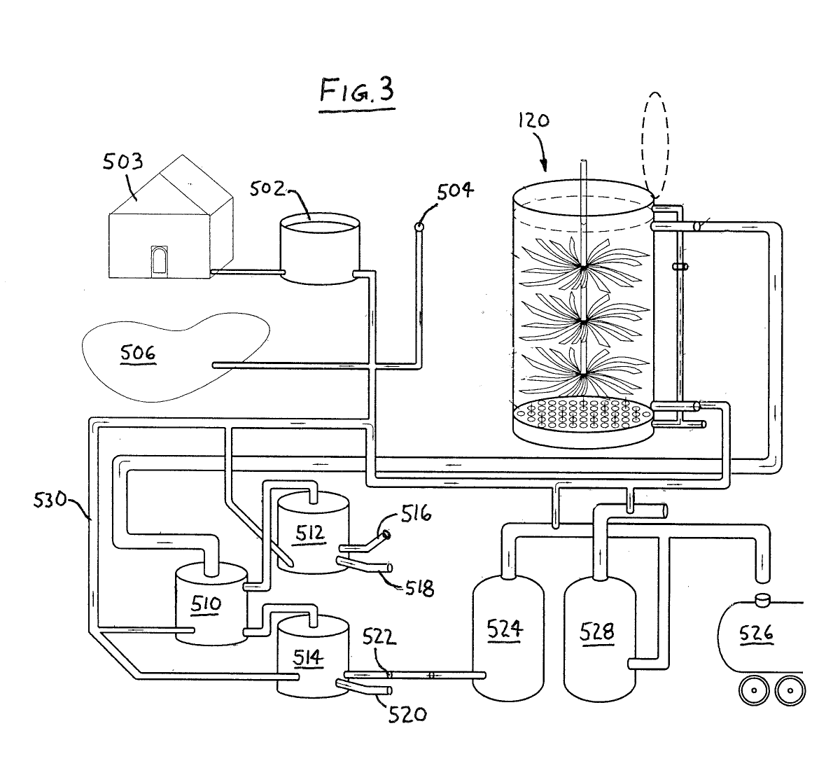

The bioreactors disclosed herein may be incorporated into a production system 500 which provides the input materials to the bioreactor and processes the algae cultivated in the bioreactor into useful products. While FIG. 3 illustrates the system 500 that uses the bioreactor 120, it will be appreciated that other bioreactors including the bioreactor 20 disclosed herein may be used without departing from the scope of the disclosure.

The water used in the bioreactor 120 may be taken from various sources. Suitable water sources include fresh and/or recycled water from a fish tank 502, sanitary sewer water 504, and storm water detention and runoff 506. Any and all of these sources may fluidly communicate with the inlet pipe 130 entering into the bioreactor 120.

When using the fish tank 502 as the water source, the fish tank 502 may fluidly communicate with a greenhouse 503 for vegetable or plant production. Fish in the fish tank 502 fertilize the algae used as feedstock in the bioreactor. Runoff from the greenhouse 503 may include nutrients for the algae that are detrimental to the fish, such as nitrogen. Nitrogen may be supplied to the bioreactor with the algae feedstock and is subsequently consumed during cultivation. As a result, a symbiotic system may be provided where the bioreactor removes nitrogen from the water in the fish tank while the fish fertilize the algae feedstock.

Any existing source of carbon dioxide may be coupled to the gas inlet pipe 154. For example, the carbon dioxide source may be a waste by-product from a separate process (list some possible sources of carbon dioxide). Carbon dioxide may also be recirculated from the tank top end 128 to the tank bottom end 126 through the gas recirculation pipe 156.

Fully cultivated algae may be pumped through the tank outlet pipe 122 to a separation tank 510. The separation tank 510 separates the algae into a lipid component and a polysaccarides protein component, wherein each component is pumped to an associated tank 512, 514. The lipids are piped to a transesterfication unit 512 which uses an ultrasonic process to create biodiesel which is pumped through outlet 516 and glycerin which is pumped through outlet 518. The polysaccharides protein is piped to a hydraulics unit 514 which uses an ultrasonic process to produce proteins which exit through outlet 520 and mono sugars which exit through outlet 522. The mono sugars may be piped to a holding tank 524 where they are fermented into ethanol or other alcohol compound. The ethanol from the fermenting tank 524 may be further piped to a tanker vehicle 526 or to an additional tank 528 for further rapid fermentation processing. Throughout the process, residual water and/or algae components may be returned to the bioreactor 120 through a return pipe 530.

While only certain embodiments have been set forth, alternatives and modifications will be apparent from the above description to those skilled in the art. These and other alternatives are considered equivalents and within the scope of this disclosure and the appended claims.

What is claimed is:

1. A bioreactor for cultivating photosynthetic organisms, comprising:
a primary tank including a sidewall oriented along a longitudinal axis;
a mixer disposed inside the tank;
a light source disposed inside the tank; and
a sparger disposed inside the tank and adapted to fluidly communicate with a source of carbon dioxide.

2. The bioreactor of claim 1, in which the primary tank sidewall is cylindrical.

3. The bioreactor of claim 2, in which the light source comprises at least one curved panel coupled to an interior surface of the sidewall.

4. The bioreactor of claim 1, in which the mixer comprises a shaft and a plurality of rotating blades coupled to the shaft.

5. The bioreactor of claim 4, in which the plurality of rotating blades are coupled to the shaft at specific elevations to create discrete sets of rotating blades.

6. The bioreactor of claim 5, in which each rotating blade comprises at least first and second sections, and in which each rotating blade first section is configured to generate fluid flow in a first axial direction and each rotating blade second section is configured to generate fluid flow in a second, opposite axial direction.

7. The bioreactor of claim 6, in which each rotating blade further comprises a third section, and in which each rotating blade third section is configured to generate fluid flow in one of the first and second axial directions.

8. The bioreactor of claim 5, in which the mixer further comprises a plurality of non-rotating blades coupled to an interior surface of the primary tank sidewall at specific elevations to create discrete sets of non-rotating blades.

9. The bioreactor of claim 8, in which the sets of rotating blades and non-rotating blades alternate along the longitudinal axis.

10. The bioreactor of claim 8, in which each non-rotating blade comprises at least first and second sections, and in which each non-rotating blade first section is configured to generate fluid flow in a first axial direction.

11. The bioreactor of claim 8, in which the light source comprises a plurality of individual lights disposed on at least some of the non-rotating blades.

12. The bioreactor of claim 1, in which the gas source comprises a plurality of gas nozzles disposed inside the tank.

13. The bioreactor of claim 12, in which at least some of the nozzles are positioned to direct a gas jet toward the light source.

14. The bioreactor of claim 1, in which the light source comprises a plurality of individual lights disposed inside the primary tank.

15. A bioreactor for cultivating photosynthetic organisms disposed in a fluid, comprising:
a primary tank including a sidewall oriented along a longitudinal axis and defining an inlet end and an outlet end;
an inlet pipe coupled to the primary tank inlet end;
an inlet valve disposed in the inlet pipe and movable between open and closed positions;
an outlet pipe coupled to the primary tank outlet end;
an outlet valve disposed in the outlet pipe and movable between open and closed positions;
a recirculation pipe having a first end coupled to the primary tank inlet end and a second end coupled to the primary tank outlet end;
a recirculation pump disposed in the recirculation pipe;
a light source disposed in at least one of the recirculation pipe and the primary tank; and
a gas source disposed inside the tank.

16. The bioreactor of claim 15, in which the bioreactor has an agitation mode in which the inlet and outlet valves are placed in the closed position and the recirculation pump is operated to agitate the fluid.

17. The bioreactor of claim 15, in which the longitudinal axis is substantially horizontal.

18. The bioreactor of claim 17, in which the primary tank is at least partially disposed underground.

19. A method for agitating photosynthetic organisms in a fluid disposed within a bioreactor, comprising:
providing a primary tank including a sidewall oriented along a longitudinal axis;
providing a mixer disposed inside the tank;
providing a light source inside the tank;
providing a gas source inside the tank;
operating the mixer to create a complex fluid flow pattern inside the primary tank, in which the complex fluid flow pattern includes at least a first and second fluid path sections, wherein the first fluid path section flows substantially in a first direction along the longitudinal axis and the second fluid path section flows substantially in a second, opposite direction along the longitudinal axis.

20. The method of claim 19, in which the complex fluid flow pattern further has a third fluid path section flowing substantially in either the first or second direction along the longitudinal axis.
